(19) United States
(12) Patent Application Publication
(10) Pub. No.: US 2021/0045458 A1
(43) Pub. Date: Feb. 18, 2021

(54) SEXUAL HEALTH GARMENTS AND METHODS OF USING THE SAME

(71) Applicant: Luxe Ventures LLC, Indianapolis, IN (US)

(72) Inventor: Michelle Janson, Indianapolis, IN (US)

(73) Assignee: Luxe Ventures LLC, Indianapolis, IN (US)

(21) Appl. No.: 16/963,475

(22) PCT Filed: Jan. 21, 2019
(86) PCT No.: PCT/US19/14431

§ 371 Date: Jul. 20, 2020

Related U.S. Application Data
(60) Provisional application No. 62/619,290, filed on Jan. 19, 2018.

(51) Int. Cl. A41B 9/12; A41B 17/00; A41B 9/04; A41B 9/02

(52) U.S. Cl. CPC A41B 9/12; A41B 17/00; A41B 9/04; A41B 2300/328; A41B 2300/32; A41B 2300/324; A41B 9/023

(57) ABSTRACT

Sexual health garments and methods of using the same. The present disclosure includes disclosure of a protective garment, comprising a front garment portion, a rear garment portion, and a non-permeable material portion extending between the front and rear garment portions when connected thereto and comprising a non-permeable material, wherein the protective garment comprises a permeable material and the non-permeable material. In a protective garment embodiment, the front garment portion and the rear garment portion form a unitary band, and wherein the non-permeable material portion extends from a relative front of the band to a relative rear of the band when connected thereto. When worn by a first person, the garment can prevent transmission of a sexually transmitted disease or infection of the first person to a second person (and vice versa) when the second person is orally stimulating the first person by oral contact with the non-permeable material portion.

### PRIORITY

The present international patent application is related to, and claims the priority benefit of, U.S. Provisional Patent Application Ser. No. 62/619,290, filed Jan. 19, 2018, the contents of which are incorporated into this disclosure directly and by reference in their entirety.

### BACKGROUND

A strong connection between head, neck, and throat cancers and oral sexual activity has been identified. Statistics from the U.S. Centers for Disease Control and Prevention (CDC) and other reputable sources continue to associate an increase in head, neck, and throat cancer to unprotected oral sexual activity. Future projections on how these types of cancers are expected to grow are alarming.

There appears to be a clear gap in the need for marketed products that can provide protection from sexually transmitted diseases (STDs) and/or sexually transmitted infections (STIs) and other cancers/diseases during sexual activity. Condoms work well for males; however, there does not appear to be anything that is simple, subtle, convenient, and mainstream for women's use. The closest option females have are dental dams and, unfortunately, based on information found via various sources, are not frequently used due to “awkwardness” and “unattractiveness” of said products. According to the CDC, there are very few options for women regarding oral sex, limited to condoms and dental dams, such as cutting the same to form a small barrier between the mouth and the partner's vagina or anus.

Harvard Medical School (HMS) is yet another reputable source highlighting the concerns of head, neck, and throat cancers being tied to oral sex, yet not suggesting any other solution regarding how to create a barrier during the sexual activity. HMS acknowledges that human papillomavirus-caused (HPV-caused) mouth and throat cancers are becoming more common, suggesting vaccinations for HPV and the use of condoms and dental dams, noting further that oral sex and “deep kissing” can transmit HPV.

In view of the same, there appears to be a clear gap in the market that, if addressed, could result in both men and women being protected from what has grown into a public health crisis and epidemic. Such a product to address the same would be well received in the marketplace.

### BRIEF SUMMARY

The present disclosure includes disclosure of a protective garment, comprising a front garment portion, a rear garment portion, and a non-permeable material portion extending between the front garment portion and the rear garment portion when connected thereto and comprising a non-permeable material, wherein the protective garment comprises a permeable material and the non-permeable material. In at least one protective garment embodiment, the non-permeable material is selected from the group consisting of latex, nitrile, rubber, polyethylene, and polyurethane. In at least one protective garment embodiment, the non-permeable material is stretchable. In at least one protective garment embodiment, the front garment portion and the rear garment portion are connected to the non-permeable material portion using a fastener. In at least one protective garment embodiment, the fastener is selected from the group consisting of stitches, hook and loop fastener, and glue. In at least one protective garment embodiment, the fastener is selected from the group consisting of a snap, a button, and tape. In at least one protective garment embodiment, the front garment portion and the rear garment portion each comprise the permeable material. In at least one protective garment embodiment, the permeable material is selected from the group consisting of cotton, polyester, satin, and silk. In at least one protective garment embodiment, the permeable material is selected from the group consisting of elastic cotton, elastic lace, and an elastic band. In at least one protective garment embodiment, it is configured to be washed and re-used. In at least one protective garment embodiment, it further comprises a liner configured to be fastened to the non-permeable material portion on a relative inside of the protective garment, the liner comprising a comfortable material. In at least one protective garment embodiment, the liner is configured to move between a first configuration covering relatively more of the non-permeable material portion and a second configuration covering relatively less of the non-permeable material portion. In at least one protective garment embodiment, it has at least one decorative element thereon. In at least one protective garment embodiment, the non-permeable material portion forms a pouch configured to retain a penis and a scrotum of a wearer of the protective garment. In at least one protective garment embodiment, the pouch is configured to stretch to accommodate the penis when erect and sufficiently cover and retain the same. In at least one protective garment embodiment, the pouch comprises a material that is relatively thinner or relatively more compliant than the non-permeable material. In at least one protective garment embodiment, it is formed by folding a unitary component at a first location, and using one or more fasteners at two additional locations to form the protective garment.

In at least one protective garment embodiment, the first location is adjacent a relative first side of the protective garment, and wherein the two additional locations comprise an opposite second side of the protective garment and a relative bottom of the protective garment. In at least one protective garment embodiment, the first location is adjacent a relative bottom of the protective garment, and wherein the two additional locations comprise a first side of the protective garment and an opposite second side of the protective garment. In at least one protective garment embodiment, it configured as panties, briefs, or boxer briefs. In at least one protective garment embodiment, the front garment portion and the rear garment portion are connected to the non-permeable material portion using heat sealing. In at least one protective garment embodiment, the front garment portion and the rear garment portion form a unitary band, and wherein the non-permeable material portion extends from a relative front of the band to a relative rear of the band when connected thereto. In at least one protective garment embodiment, the front garment portion and the rear garment portion comprise the permeable material. In at least one protective garment embodiment, the permeable material comprises cotton, and wherein the non-permeable material is selected from the group consisting of latex and nitrile. In at least one protective garment embodiment, the unitary band comprises the permeable material comprising elastic material, in at least one protective garment embodiment, the non-permeable material portion is reversibly coupled to the unitary band using a fastener selected from the group consisting of tape and an adhesive. In at least one protective garment embodiment, the permeable material and the non-permeable material are stretchable. In at least one protective garment embodiment, the permeable material and the non-permeable material stretch to different extents. In at least one protective garment embodiment, the unitary band has a width at or between 1″ and 1.5.″ In at least one protective garment embodiment, the unitary band is sized and shaped to cover a wearer's navel when the protective garment is worn by the wearer. In at least one protective garment embodiment, the non-permeable material portion comprises a first fastener at or near a first end and a second fastener at or near a second end, the first fastener configured to couple to the unitary band at a relative front of the unitary band, and the second fastener configured to couple to the unitary band at a relative rear of the unitary band.

In at least one protective garment embodiment, the non-permeable material portion has indicia thereon to identify a location of at least one fastener. In at least one protective garment embodiment, the non-permeable material portion is rectangular in shape. In at least one protective garment embodiment, the non-permeable material portion is tapered inward at each relative side so that a wearer's inner legs engage the non-permeable material portion at the tapered relative sides when the protective garment is worn by the wearer. In at least one protective garment embodiment, the unitary band is a partial garment, and wherein the protective garment forms part of a kit, the kit further comprising at least a second non-permeable material portion. In at least one protective garment embodiment, the unitary band is a partial garment, and wherein the protective garment forms part of a kit, the kit further comprising at least a second non-permeable material portion, a third non-permeable material portion, and a second partial garment, wherein the second non-permeable material portion and the third non-permeable material portion each have at least one fastener coupled thereto. In at least one protective garment embodiment, the non-permeable material portion has an opening defined therein, the opening sized and shaped to allow a penis of a wearer to extend through the opening while the non-permeable material portion remains taught around the penis. In at least one protective garment embodiment, the non-permeable material portion has an opening defined therein, the opening sized and shaped to allow a penis of a wearer to extend through the opening while the non-permeable material portion remains taught around the penis. In at least one protective garment embodiment, the opening is sized and shaped to allow the penis covered in a condom to extend through the opening while the non-permeable material portion remains taught around the penis. In at least one protective garment embodiment, it forms part of a kit, the kit further comprising a second non-permeable material portion. In at least one protective garment embodiment, it forms part of a kit, the kit further comprising a second non-permeable material portion. In at least one protective garment embodiment, when worn by a first person, is configured to prevent fluid or other matter from a vagina, a penis, and/or an anus of the first person from transferring to the second person when the second person orally stimulates the first person by oral contact with the non-permeable material portion. In at least one protective garment embodiment, when worn by a first person, is configured to prevent transmission of a sexually transmitted disease or a sexually transmitted infection of the first person to a second person who is orally stimulating the first person by oral contact with the non-permeable material portion. In at least one protective garment embodiment, when worn by a first person, is configured to prevent transmission of a sexually transmitted disease or a sexually transmitted infection of a second person to the first person when the second person is orally stimulating the first person by oral contact with the non-permeable material portion.

The present disclosure includes disclosure of a protective garment, comprising a front garment portion connected to a rear garment portion, and a non-permeable material portion extending between the front garment portion and the rear garment portion and comprising a non-permeable material, wherein the protective garment comprises a permeable material and the non-permeable material, wherein the permeable material is selected from the group consisting of cotton and elastic, wherein the non-permeable material is selected from the group consisting of rubber, latex, and nitrile, and wherein when the protective garment is worn by a first person, fluid or other matter from a vagina, a penis, and/or an anus of the first person is prevented from transferring to a second person when the second person is orally stimulating the first person by oral contact with the non-permeable material portion.

The present disclosure includes a method, comprising a first person wearing a protective garment of the present disclosure so that the non-permeable material portion is adjacent a vagina, a penis, a scrotum, and/or an anus of the first person, and a second person orally stimulating the first person by placing lips and/or a tongue of the second person on the protective garment at the non-permeable material portion. In at least one method, fluid or other matter from the vagina, the penis, and/or the anus of the first person is prevented from transferring to the second person (and vice versa) when the second person is performing the step of orally stimulating the first person.

### BRIEF DESCRIPTION OF TIM DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a front view of a protective garment, according to at least one embodiment of the present disclosure;

FIG. 2 shows a rear/back view of a protective garment, according to at least one embodiment of the present disclosure;

FIG. 3 shows a front perspective open view of a protective garment, according to at least one embodiment of the present disclosure;

FIG. 4 shows a rear/back perspective open view of a protective garment, according to at least one embodiment of the present disclosure;

FIGS. 5 and 6 show opposing side perspective open views of a protective garment, according to at least one embodiment of the present disclosure;

FIG. 7 shows a top open view of a protective garment, according to at least one embodiment of the present disclosure;

FIG. 8 shows a protective garment comprising all (or nearly all) non-permeable material, according to at least one embodiment of the present disclosure;

FIG. 9, FIG. 10, and FIG. 11 show embodiments of a unitary component, according to embodiments of the present disclosure;

FIG. 12 shows a front perspective open view of a protective garment having a pouch, according to at least one embodiment of the present disclosure;

FIG. 13 shows a side perspective open view of a protective garment having a pouch, according to at least one embodiment of the present disclosure;

FIG. 14 shows a front view of an embodiment of a protective garment with a relatively narrow front garment portion (band), according to at least one embodiment of the present disclosure;

FIG. 15 shows a front view of the embodiment of the protective garment shown in FIG. 14 being worn by a wearer, according to at least one embodiment of the present disclosure;

FIG. 16 shows a front view of an embodiment of a protective garment with a relatively wide front garment portion (band), according to at least one embodiment of the present disclosure;

FIG. 17 shows a front view of the embodiment of the protective garment shown in FIG. 16 being worn by a wearer, according to at least one embodiment of the present disclosure;

FIG. 18 shows an inside view of a non-permeable garment portion not coupled to other portions of a protective garment, according to at least one embodiment of the present disclosure;

FIG. 19 shows an inside view of a non-permeable garment portion having tapered sides and not coupled to other portions of a protective garment, according to at least one embodiment of the present disclosure;

FIG. 20 shows a block component diagram of a kit, according to at least one embodiment of the present disclosure;

FIG. 21 shows a front view of an embodiment of a protective garment with an opening for a penis defined therein, according to at least one embodiment of the present disclosure; and

FIG. 22 shows a front view of the embodiment of the protective garment shown in FIG. 21 being worn by a wearer with the wearer's penis passing through the opening, according to at least one embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified. form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

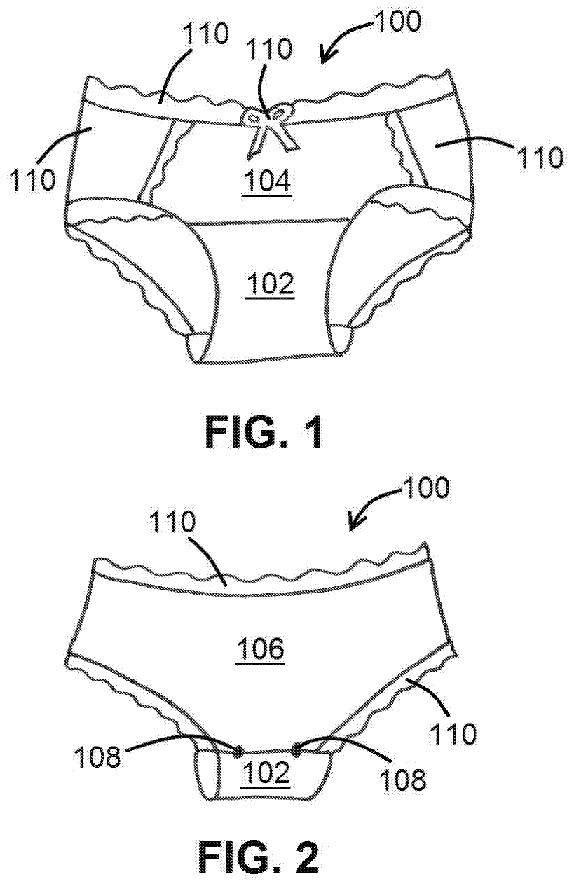

The present disclosure includes disclosure of protective garments, including protective undergarments, as provided in further detail herein. An exemplary protective garment of the present disclosure is shown in FIGS. 1 and 2. FIG. 1 shows a front view of an exemplary protective garment 100 of the present disclosure, and FIG. 2 shows a rear/back view of an exemplary protective garment 100 of the present disclosure. As shown in FIGS. 1 and 2, an exemplary protective garment 100 of the present disclosure can be configured as traditional underwear, such as panties, briefs, boxer briefs, and the like, whereby protective garment 100 comprises a non-permeable material portion 102 extending between a front garment portion 104 and a rear garment portion 106. Non-permeable material portion 102 can comprise any number of relatively thin, non-permeable materials, such as latex, nitrile, rubber, one or more polyethylenes, polyurethane, other organic materials, and the like, that can be coupled to front garment portion 104 and rear garment portion 106, as referenced herein. Materials such as latex, nitrile, rubber, etc., have inherent stretchability properties, making said materials quite suitable for use with non-permeable material portions 102 of the present disclosure.

When an exemplary protective garment 100 of the present disclosure is worn, such as by a woman, the front garment portion 104 (in the embodiments shown in FIGS. 1-8, for example, would be adjacent to a front pelvic area of the woman, the rear garment portion 106 would be adjacent to the buttocks of the woman, and the non-permeable portion 102 would be adjacent to one or both of the vagina and/or the anus (generally referred to as the crotch) of the woman. Potential oral stimulation, cunnilingus, and/or other potential sexual acts performed by the woman's partner could then be performed while the woman is wearing protective garment 100, and said acts, which could involve the use of the partner's mouth (lips and/or tongue) would be performed upon non-permeable material portion 102 so that the performer does not actually come into contact with the genital and/or anal region of the wearer of protective garment 100, but so that the wearer of protective garment 100 can feel the stimulation from said acts through the non-permeable material portion 102, and so that any potential fluids or other matter from the wearer of protective garment 100 would not conic into contact with the performer of said acts, and so that any potential fluids or other matter from the performer of said acts come into contact with the wearer of protective garment 100, due to the non-permeable nature of non-permeable material portion 102. Non-permeable material portion 102, in various embodiments, would be thin and/or flexible to allow for stimulation from said acts to be felt by the wearer of protective garment 100.

Non-permeable material portion 102, as referenced herein, can be coupled to front garment portion 104 and rear garment portion 106, such as by stitching/sewing non-permeable material portion 102 thereto, or by way of another coupling mechanism, such as one or more snaps, buttons, hook and loop fasteners (such as Velcro®), glues (adhesives), tapes (such as garment tape or other tapes), etc., collectively referred to herein as an optional fastener 108, or by heat-sealing, for example, which effectively binds to portions together due to heat. Front garment portion 104 and rear garment portion 106 can each comprise cloth fabric (cotton, polyester, blends, etc.), satin, silk, elastic (or other stretchable material, including but not limited to elastic cotton, elastic lace, or other stretchable materials, including elastic bands), rubber, nylon, spandex, polypropylene, etc., and can be permeable (comprising a permeable material) as may be desired. For example, and in at least one embodiment of the present disclosure, protective garment 100 comprises a front garment portion 104 and a rear garment portion 106 made of cotton, and non-permeable material portion 102 can be made of latex or nitrile, for example.

Exemplary protective garments 100 of the present disclosure can be configured for one-time use, or can be configured to be laundered or otherwise washed, as may be desired. Protective garments 100 of the present disclosure could be worn for an evening/duration of time or potentially only when a sexual act is to occur.

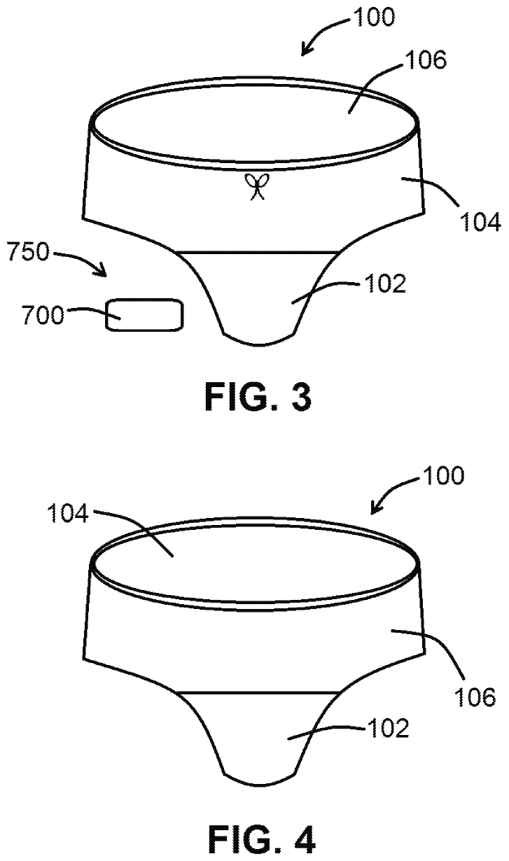

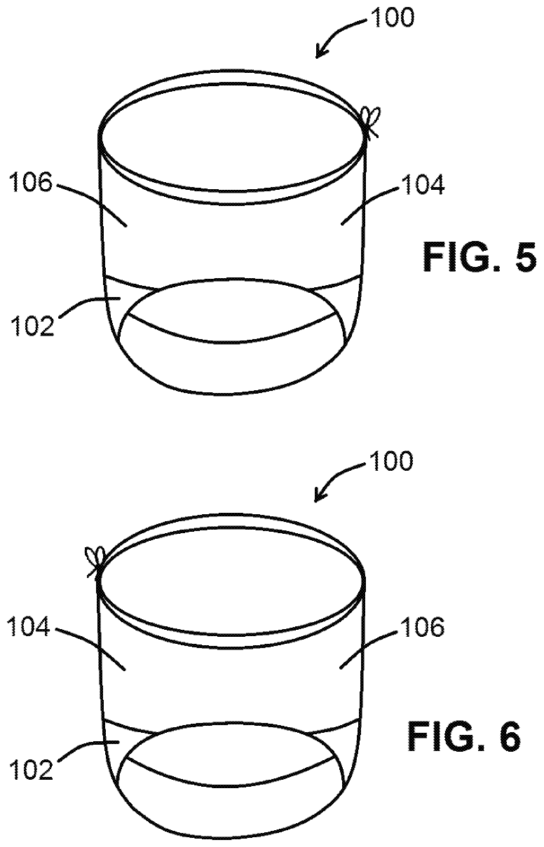

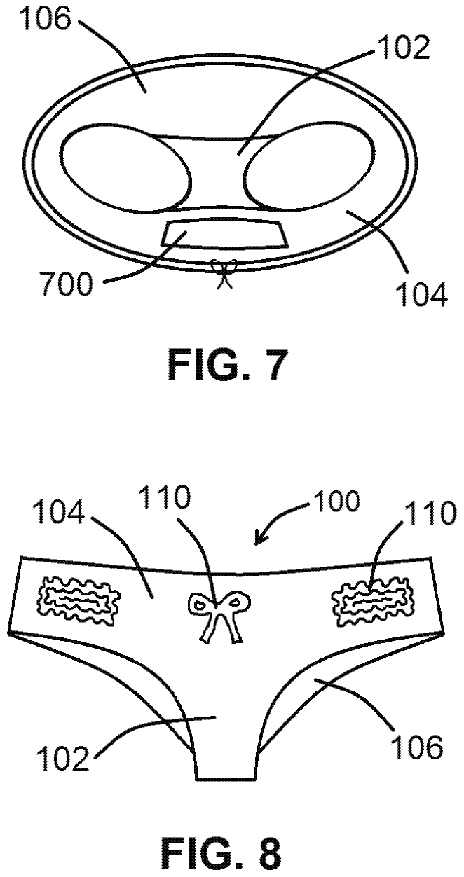

FIG. 3 shows a front perspective open view, and FIG. 4 shows a rear/back perspective open view, of an exemplary protective garment 100 of the present disclosure. FIGS. 5 and 6 show opposing side open views, and FIG. 7 shows a top open view of an exemplary protective garment 100 of the present disclosure.

Exemplary protective garments 100 of the present disclosure may also comprise a liner 700, such as a removable liner 700 or a liner 700 that is attached to protective garment 100, such as being stitched/sewn, snapped, buttoned, or otherwise fastened to protective garment 100. Such an exemplary liner 700 is shown in FIG. 7, whereby liner 700 is attached to protective garment 100 so that it can fold down (noting that liner 700 is shown adjacent to a relative front garment portion 104 in FIG. 7, so to be folded down to cover non-permeable material portion 102) or otherwise be positioned over non-permeable material portion 102 within protective garment 100. Liners 700 can comprise any number of materials, such as cotton, polyester, blends, satin, silk, etc., and are intended to provide comfort to the wearer (referred to herein as a comfortable material) of protective garment 100 during times of no sexual activity. Liner 700 can be repositioned (such as folded upward) or removed when sexual activity is to occur, as may be desired. As such, a wearer of protective garment 100 throughout an evening can use a liner 700 upon the non-permeable material portion 102 and prevent any potential discomfort or irritation of latex/similar material (the non-permeable material portion 102) from direct skin contact until or before sexual activity is to occur. If a liner 700 is not affixed to protective garment 100 but is instead merely positioned adjacent to protective garment 100, the combination of a protective garment 100 and one or more liners 700 can be referred to as a garment system 750 of the present disclosure, such as shown in FIG. 3.

Protective garments 100 of the present disclosure can also have one or more decorative elements 110, such as lace panels, lace ruffles, other panels/ruffles, bows, indicia (decoration(s), logo(s), and the like), etc., such as shown in FIG. 1. Protective garments 100 of the present disclosure can comprise various materials, colors, sizes, patterns, and/or accents, as may be desired.

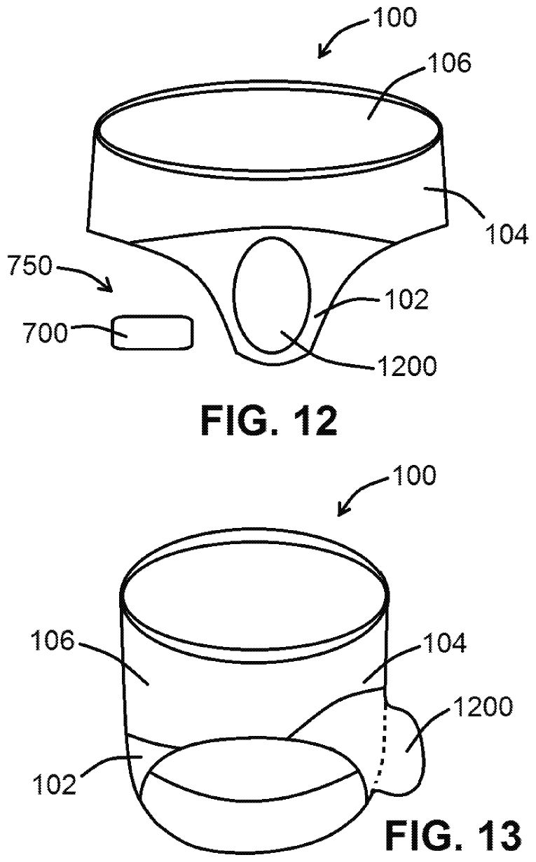

Protective garments 100 of the present disclosure may be worn by any gender. The disclosure herein provides several examples/instances of women wearing protective garments 100, such as when receiving oral stimulation to the vagina or anus. However, protective garments 100 of the present disclosure can also be worn by men, whereby the non-permeable material portion 102 can cover the penis, scrotum/testicles, and anus so that oral stimulation to the same would be protected by said barrier (non-permeable material portion 102). In such embodiments, non-permeable material portion 102 could form a pouch 1200 (such as shown in FIGS. 12 and 13) in a relative front and/or relative bottom of the protective garment 100, so to allow for stretching for an erect penis and still adequately cover the same. As such, protective garments 100 of the present disclosure can be configured as panties, briefs, boxer briefs, etc., to be worn by men or women, and by individuals of any gender or orientation, such as individuals who are lesbian, gay, bisexual, transgender, queer, or identified in related communities (LGBTQ+).

As referenced herein, exemplary protective garments 100 of the present disclosure can be considered as novel modified underwear having a very thin, non-permeable latex (or equivalent) crotch (the non-permeable material portion 102) that would be worn during oral stimulation of the vagina, penis, scrotum/testicles, and/or anus. Protective garments 100 of the present disclosure are intended to support sexual health. Said protective garments 100 are intended for use by sexually active adults, noting that the CDC has identified that the majority of adults have engaged in oral sex, also acknowledging that a significant percentage of teenage girls and boys have also engaged in oral sex. In view of the same, protective garments 100 of the present disclosure can be used during oral sex, and can therefore reduce the likelihood/risk of transmitting fluids, bacteria, STDs, STIs, or other matter from one partner to the other, or even prevent the transmission of fluids, bacteria, STDs, STIs, or other matter from one partner to the other, when one partner is wearing a protective garment 100 of the present disclosure, due to the presence and location of non-permeable material portion 102 relative to the vagina and/or anus of the wearer of said protective garment 100, for example. As such, protective garments 100 of the present disclosure are considered as effective sexual health products.

FIG. 8 shows an embodiment of a protective garment 100 of the present disclosure, which comprises only non-permeable material, so that the non-permeable portion 102 is effectively the entire protective garment 100. As such, front garment portion 104 and rear garment portion would comprise the non-permeable material in addition to the non-permeable portion 102 as protective garment 100 would be made of the non-permeable material. One or more decorative elements 110 can be printed on or otherwise affixed to protective garment 100, such as lace panels, lace ruffles, other panels/ruffles, bows, indicia, etc., so that protective garment 100 is still considered as being completely made of the non-permeable material, but having optional decorative elements 110 printed thereon or otherwise affixed thereto. For example, and as shown in FIG. 8, indicia (an exemplary decorative element 110) can be positioned at one or more areas of protective garment 100.

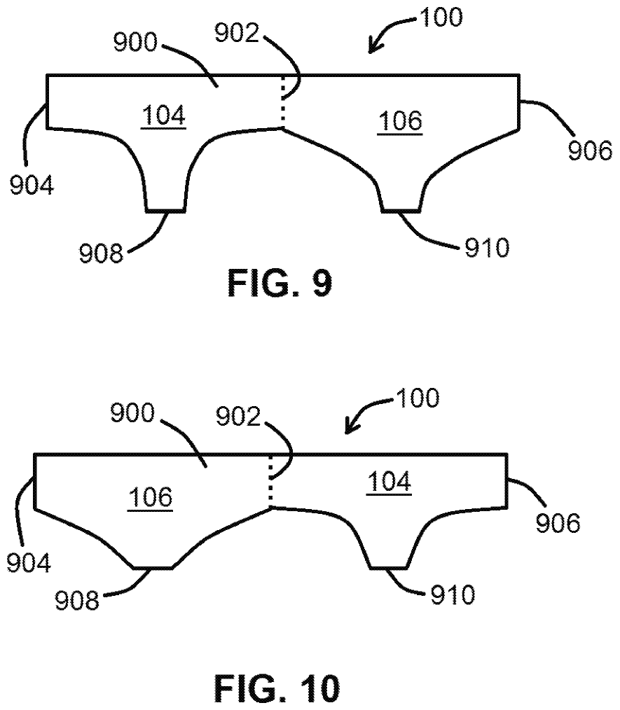

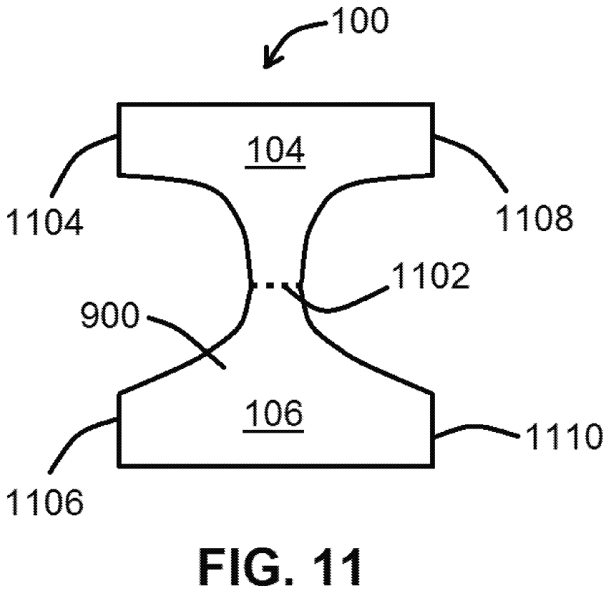

FIGS. 9, 10, and 11 show exemplary protective garments 100 in non-finished forms. For example, and a shown therein, a unitary component 900 can comprise a relative front garment portion 104 and a relative rear garment portion 106, whereby folding said unitary component 900 can facilitate final connection of one or more elements of said unitary component 900 to form a wearable protective garment 100.

FIG. 9 shows a unitary component 900 having the relative front garment portion 104 on a relative left side and a relative rear garment portion 106 on a relative right side. Folding the unitary component at fold 902 (whereby fold 902 is at a relative middle of unitary component 900 at relative side of protective garment 100 when folded and worn) would or could ultimately result with left edge 904 being adjacent to right edge 906, and ultimately result in front bottom edge 908 being adjacent to rear bottom edge 910, so that left edge 904 could be coupled to right edge 906 and so that front bottom edge 908 could be coupled to rear bottom edge 910, such as by heat-sealing, gluing, stitching, taping, etc. (an exemplary fastener 108) said edges to one another to form a wearable protective garment 100.

FIG. 10 shows a unitary component 900 having the relative rear garment portion 106 on a relative left side and a relative front garment portion 104 on a relative tight side. Folding the unitary component at fold 902 (whereby fold 902 is at a relative middle of unitary component 900 at relative side of protective garment 100 when folded and worn) would or could ultimately result with left edge 904 being adjacent to right edge 906, and ultimately result in front bottom edge 908 being adjacent to rear bottom edge 910, so that left edge 904 could be coupled to right edge 906 and so that front bottom edge 908 could be coupled to rear bottom edge 910, such as by heat-sealing, gluing, stitching, taping, etc. (an exemplary fastener 108) said edges to one another to form a wearable protective garment 100.

FIG. 11 shows a unitary component 900 having the relative front garment portion 104 and the relative rear garment portion 106 formed together, whereby folding unitary component 900 at fold 1102 (whereby fold 1002 is at a relative middle of unitary component at relative bottom of protective garment 100 when folded and worn) would or could ultimately result with front portion first edge 1104 being adjacent to rear portion first edge 1106, and ultimately result in front portion second edge 1108 being adjacent to rear portion second edge 1110, so that left portion first edge 1104 could be coupled to rear portion first edge 1106 and so that front bottom portion second edge 1108 could be coupled to rear portion second edge 1110, such as by heat-sealing, gluing, stitching, taping, etc. (an exemplary fastener 108) said edges to one another to form a wearable protective garment 100.

FIGS. 12 and 13 show embodiments of a protective garment 100 of the present disclosure, whereby the non-permeable material portion 102 (noting that, for example, the entire protective garment 100 could comprise said material or part of said garment 100) positioned at a relative front and/or relative bottom of protective garment 100 (such as when worn) has a pouch 1200 formed therein, pouch 1200 providing additional comfort to a wearer's penis 2200 (as shown in FIG. 22) and/or scrotum/testicles. Pouch 1200 can comprise a portion of said non-permeable material portion 102 that is formed so that said portion of non-permeable material portion 102 includes additional and/or thinner material and/or more compliant material to allow protective garment 100 to stretch more than it could stretch without said pouch 1200, such as to allow a wearer to remain comfortable while wearing protective garment 100 with an erection.

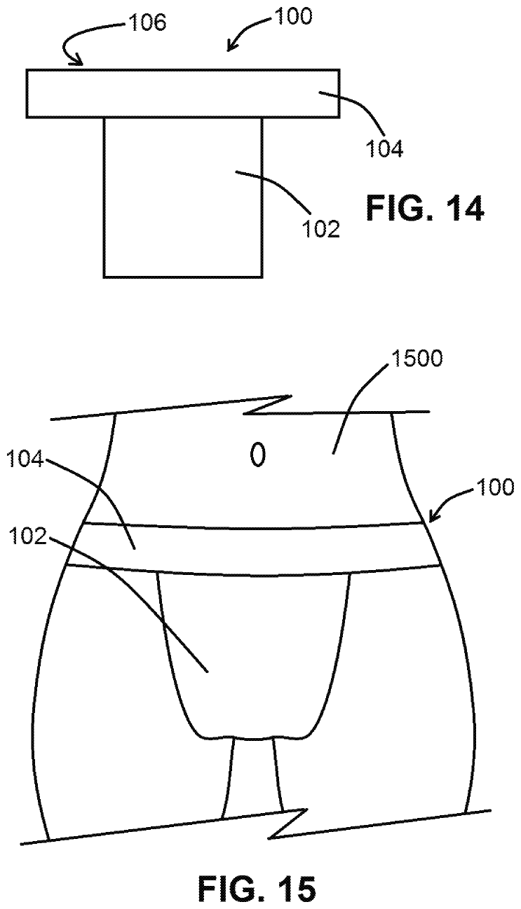

FIG. 14 shows an embodiment of a protective garment 100 having a non-permeable material portion 102, a front garment portion 104, and a rear garment portion 106, such as generally described herein. Front garment portion 104 and rear garment portion 106 are relative portions of a protective garment 100 of the present disclosure, and can be separate elements or one single element, as may be desired. As shown in FIG. 14, such as also shown in FIGS. 3 and 4 for example, front garment portion 104 and rear garment portion 106 can be regions or portions of the same overall element, such as one piece of fabric or elastic (an elastic band, for example). Non-permeable material portion 102, as shown in FIG. 14 and similarly as shown in FIGS. 3 and 4, connects or couples to front garment portion 104 at a relative front of the garment 100 (such as at a relative front of the elastic band, for example) and connects to a rear garment portion 106 at a relative rear of the garment 100 (such as at a relative rear of the elastic band, for example), such as described herein in various embodiments, by heat-sealing, gluing (use of an adhesive), stitching, taping, etc. (use of an exemplary fastener 108). In at least one embodiment, front garment portion 104 and rear garment portion 106 are a single component made of elastic and/or another stretchable material (a “first” material), and non-permeable material portion 102 is a component made of a non-permeable material that is also stretchable, such as rubber or latex (a “second” material), whereby the first material is either the same material (so to stretch to the same extent) or a different material from/than the second material (so to stretch to different extents). Exemplary front garment portions 104, such as shown in FIG. 14, can be at or about 1½″ tall/wide, or thinner or wider as may be desired, such as at or between 1″ and 1.5″ tall/wide.

FIG. 15 shows an embodiment of a protective garment 100 of the present disclosure being worn by a person 1500. As shown therein, protective garment 100 covers the genital and/or anal region of the person 1500, and the flexibility/elasticity of one or more materials comprising the non-permeable material portion 102, the front garment portion 104, and/or the second garment portion 106, results in a comfortable and effective protective garment 100.

In at least some embodiments of protective garments 100 of the present disclosure, the non-permeable material portion 102, when coupled or connected to a front garment portion 104 and a rear garment portion 106, is connected using an exemplary fastener 108 of the present disclosure that permits a first non-permeable material portion 102 to be used, removed, and replaced by a replacement non-material portion 102 or the same non-permeable material portion 102, such as after washing said used portion(s). Such an embodiment of a protective garment 100 could allow a user to reuse portions of said garment 100 and replace portions of said garment 100 as may be desired.

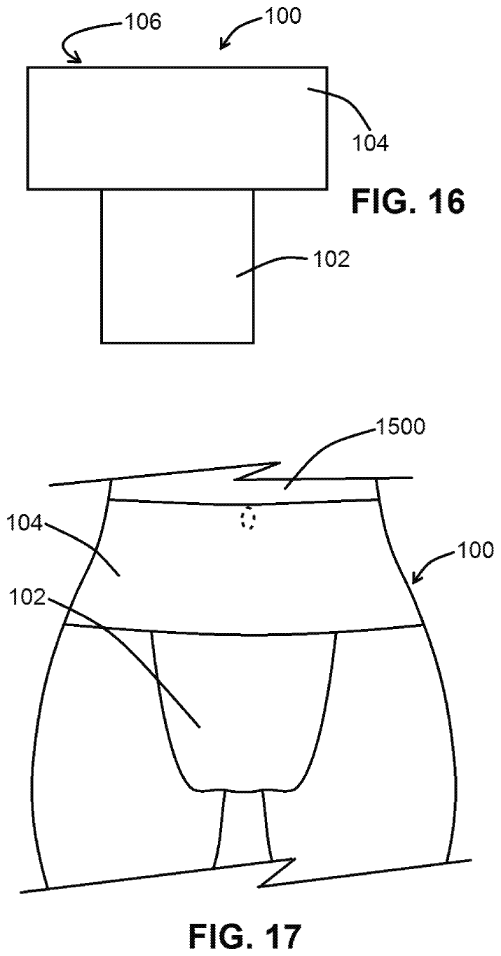

FIGS. 16 and 17 depict embodiments of a protective garment 100 of the present disclosure, whereby the front garment portion 104 and optionally the rear garment portion 106 are relatively tall/wide (as compared to the embodiments shown in FIGS. 15 and 16, for example), to provide additional comfort, to help to slim the abdominal area when protective garment 100 is worn, and to help with the wearer's overall appearance and/or confidence when wearing said protective garment 100. Front garment portion 104, in at least one embodiment, is configured to cover a wearer's navel, such as shown in FIG. 15, and/or more of the wearer's hip, buttocks, pubic area, and/or genital areas. Non-permeable material portions 102 could be the same size as used with other embodiments, or could be relatively larger or smaller, such as being smaller in embodiments whereby the front garment portion 104 is configured to cover some or all of the wearer's, buttocks, hip, pubic, and/or genital areas. Exemplary front garment portions 104, such as shown in FIG. 16, can be at or about 6″ tall/wide, or thinner or wider as may be desired, such as at or between 5″ and 7″ tall/wide.

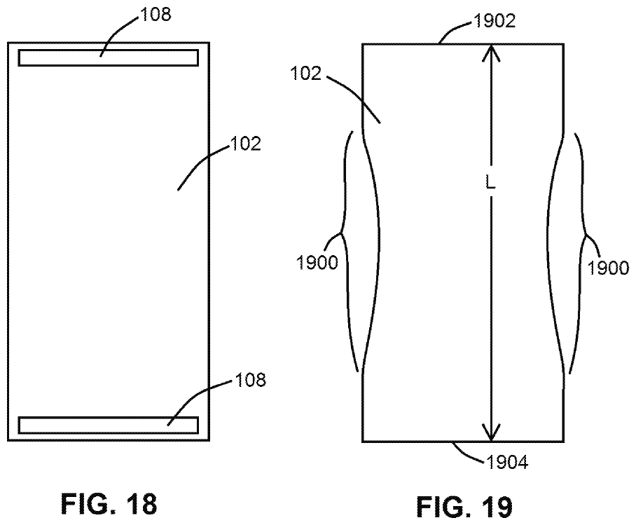

FIG. 18 shows an exemplary non-permeable material portion 102 of the present disclosure not coupled to a front garment portion 104 or a rear garment portion 106. As shown therein, fasteners 108 (such as snaps, buttons, portions of hook and loop fasteners (such as Velcro®), glues, tapes (such as garment tape or other tapes), etc., can be positioned upon or within non-permeable material portion 102, so that said fasteners 108 can be used to connect/couple non-permeable material portion 102 to a front garment portion 104 and a rear garment portion 106 to form an exemplary protective garment 100 of the present disclosure. FIG. 19 shows an exemplary non-permeable material portion 102 of the present disclosure not coupled to a front garment portion 104 or a rear garment portion 106 and not comprising any fasteners 108. Exemplary non-permeable garment portions 102 can be 6″ wide×18″ long, 7″ wide×20″ long, or wider or narrower and/or longer or shorter, as may be desired. If the fasteners 108 are not already coupled to or formed as part of an exemplary non-permeable material portion 102, such as shown in FIG. 18, indicia (an exemplary decorative element 110) can be positioned upon or within non-permeable material portion 102 to indicate where the fastener(s) 108 are to be positioned.

Non-permeable material portions 102, such as shown in FIG. 18, may be rectangular, relatively rectangular, or some other shape as may be desired. A tapered portion 1900 may exist on one or both sides of an exemplary non-permeable material portion 102, such as shown in FIG. 19, so to provide additional comfort to the wearer by having less material at the crotch, for example. Tapered portions 1900 (which may be curved or of a different shape) can exist or be defined within non-permeable material portion 102 and extend an entire length (“L,” extending from a first end 1902 to a second end 1904), or part of a length L of said non-permeable material portion 102, such as shown in FIG. 19.

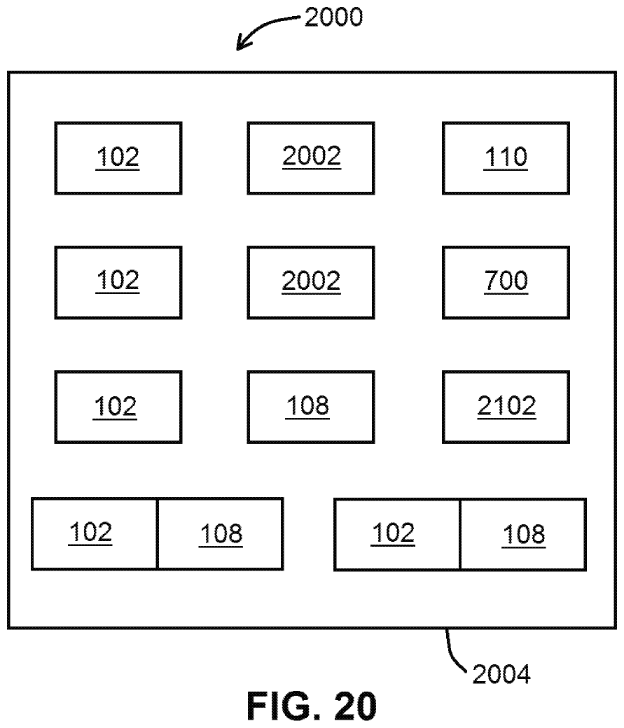

FIG. 20 shows an exemplary kit 2000 of the present disclosure in block diagram form. As shown therein, an exemplary kit 2000 of the present disclosure can comprise any number of non-permeable material portions 102 that do or do not have one or more fasteners 108 affixed thereto, formed as part thereof, or packaged therewith, and one or more partial garments 2002, whereby partial garments 2002 comprise front material portion 104 and/or rear material portion 106, as separate items or connected to one another (or formed as a unitary piece). Kits 2000 of the present disclosure can include one, two, three, four, five, or more, of one or more of the various elements referenced herein, including but not limited to non-permeable material portions 102, front material portions 104, rear material portions 106, fasteners 108, decorative elements 110, liners 700, partial garments 2002, and/or condoms 2102. Packaging 2004 can be used to house the various kit 2000 elements, with packaging being designed as desired.

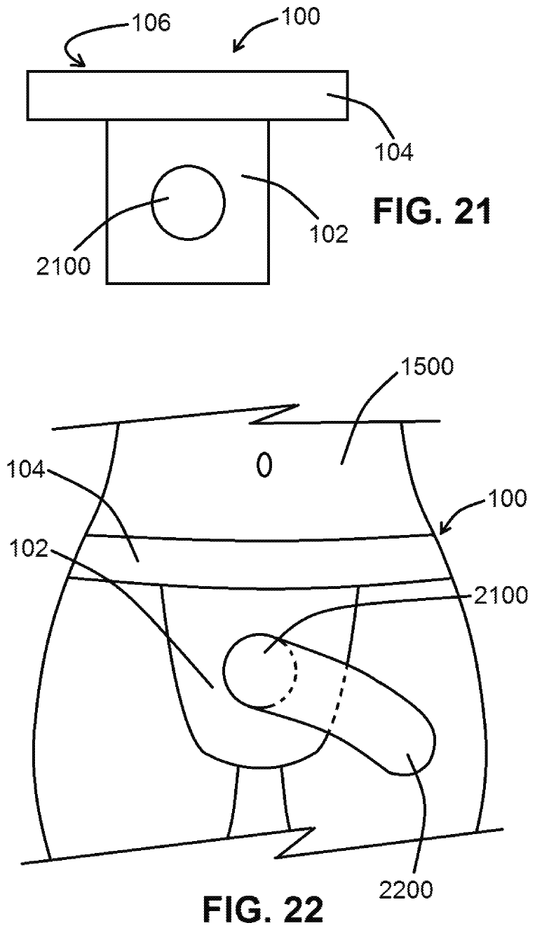

FIG. 21 shows an embodiment of a protective garment 100 having a non-permeable material portion 102, a front garment portion 104, and a rear garment portion 106, such as generally described herein. Front garment portion 104 and rear garment portion 106 are relative portions of a protective garment 100 of the present disclosure, and can be separate elements or one single element, as may be desired. As shown in FIG. 21, such as also shown in FIGS. 3, 4, 14, and 16, for example, front garment portion 104 and rear garment portion 106 can be regions or portions of the same overall element, such as one piece of fabric or elastic. Non-permeable material portion 102, as shown in FIG. 21 and similarly as shown in FIGS. 3, 4, 14, and 16, connects or couples to front garment portion 104 at a relative front of the garment 100 and connects to a rear garment portion 106 at a relative rear of the garment 100, such as described herein, by heat-sealing, gluing, stitching, taping, etc. (use of an exemplary fastener 108). In at least one embodiment, front garment portion 104 and rear garment portion 106 are a single component made of elastic and/or another stretchable material (a “first” material), and non-permeable material portion 102 is a component made of a non-permeable material that is also stretchable, such as rubber or latex (a “second” material), whereby the first material is either the same or a different material from/than the second material.

In the embodiment shown in FIG. 21, the non-permeable material portion 102 has an opening 2100 defined therein/therethrough, said opening 2100 sized and shaped to allow a penis 2200 (referenced herein as a human penis or an artificial penile device, which passes through opening 2100 or is otherwise positioned outside of non-permeable material portion 102, including being coupled thereto if desired) of penile device 2200 to pass therethrough, such as shown in FIG. 22. A wearer of protective garment 100, for example, can allow his penis 2200 (flaccid or erect) to comfortably fit through opening 2100, whereby opening 2100 is configured to comfortably receive penises 2200 of various sizes, and whereby non-permeable material portion 102 remains taught enough around penis 2200 so to provide a close fit around the base of the penis 2200. The wearer can wear a condom 2102, which can be part of a kit 2000 as shown in FIG. 20 or not as part of said kit 2000, preferably prior to wearing the protective garment 100, so that the end of the condom 2102 near the base of the penis 2200 remains in place and provides better coverage and protection of the genital region of the wearer.

While various embodiments of sexual health garments and methods of using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A protective garment, comprising:
a front garment portion;
a rear garment portion; and
a non-permeable material portion extending between the front garment portion and the rear garment portion when connected thereto;
wherein the protective garment comprises a permeable material and the non-permeable material; and
wherein the front garment portion and the rear garment portion form a unitary band, and wherein the non-permeable material portion extends from a relative front of the band to a relative rear of the band when connected thereto.

2. The protective garment of claim 1, wherein the non-permeable material is selected from the group consisting of latex, nitrile, rubber, polyethylene, and polyurethane.

3. (canceled)

4. The protective garment of claim 1, wherein the front garment portion and the rear garment portion are connected to the non-permeable material portion using a fastener selected from the group consisting of stitches, hook and loop fastener, glue, a snap, a button, and tape.

5. (canceled)

6. (canceled)

7. The protective garment of claim 1, wherein the front garment portion and the rear garment portion each comprise the permeable material, wherein the permeable material is selected from the group consisting of cotton, polyester, satin, silk, elastic lace, and an elastic band.

8. (canceled)

9. (canceled)

10. (canceled)

11. The protective garment of claim 1, further comprising a liner configured to be fastened to the non-permeable material portion on a relative inside of the protective garment, the liner comprising a comfortable material, and wherein the liner is configured to move between a first configuration covering relatively more of the non-permeable material portion and a second configuration covering relatively less of the non-permeable material portion.

12. (canceled)

13. (canceled)

14. The protective garment of claim 1, wherein the non-permeable material portion forms a pouch configured to retain a penis and a scrotum of a wearer of the protective garment, wherein the pouch comprises a material that is relatively thinner or relatively more compliant than the non-permeable material, and wherein the pouch is configured to stretch to accommodate the penis when erect and sufficiently cover and retain the same.

15. (canceled)

16. (canceled)

17. (canceled)

18. (canceled)

19. (canceled)

20. The protective garment of claim 1, having a configuration selected from the group consisting of panties, boxer briefs, and briefs.

21. (canceled)

22. (canceled)

23. The protective garment of claim 1, wherein the front garment portion and the rear garment portion are connected to the non-permeable material portion using heat sealing.

24. (canceled)

25. (canceled)

26. (canceled)

27. (canceled)

28. The protective garment of claim 1, wherein the non-permeable material portion is reversibly coupled to the unitary band using a fastener selected from the group consisting of tape and an adhesive.

29. The protective garment of claim 1, wherein the permeable material and the non-permeable material are stretchable to different extents.

30. (canceled)

31. The protective garment of claim 1, wherein the unitary band has a width at or between 1″ and 1.5″, and wherein the unitary band is sized and shaped to cover a wearer's navel when the protective garment is worn by the wearer.

32. (canceled)

33. The protective garment of claim 1, wherein the non-permeable material portion comprises a first fastener at or near a first end and a second fastener at or near a second end, the first fastener configured to couple to the unitary band at a relative front of the unitary band, and the second fastener configured to couple to the unitary band at a relative rear of the unitary band.

34. (canceled)

35. (canceled)

36. The protective garment of claim 1, wherein the non-permeable material portion is tapered inward at each relative side so that a wearer's inner legs engage the non-permeable material portion at the tapered relative sides when the protective garment is worn by the wearer.

37. (canceled)

38. The protective garment of claim 1, wherein the unitary band is a partial garment, and wherein the protective garment forms part of a kit, the kit further comprising at least a second non-permeable material portion, a third non-permeable material portion, and a second partial garment, wherein the second non-permeable material portion and the third non-permeable material portion each have at least one fastener coupled thereto.

39. The protective garment of claim 1, wherein the non-permeable material portion has an opening defined therein, the opening sized and shaped to allow a penis of a wearer to extend through the opening while the non-permeable material portion remains taught around the penis.

40. (canceled)

41. (canceled)

42. The protective garment of claim 1, forming part of a kit, the kit further comprising a second non-permeable material portion.

43. (canceled)

44. The protective garment of claim 1, when worn by a first person, is configured to prevent fluid or other matter from a vagina, a penis, and/or an anus of the first person from transferring to the second person when the second person orally stimulates the first person by oral contact with the non-permeable material portion.

45. The protective garment of claim 1, when worn by a first person, is configured to prevent transmission of a sexually transmitted disease or a sexually transmitted infection of the first person to a second person who is orally stimulating the first person, or from the second person who is orally stimulating the first person, by oral contact with the non-permeable material portion.

46. (canceled)

47. A protective garment, comprising:
a front garment portion connected to a rear garment portion; and
a non-permeable material portion extending between the front garment portion and the rear garment portion and comprising a non-permeable material;
wherein the protective garment comprises a permeable material and the non-permeable material;
wherein the permeable material is selected from the group consisting of cotton and elastic;
wherein the non-permeable material is selected from the group consisting of rubber, latex, and nitrile; and
wherein when the protective garment is worn by a first person, fluid or other matter from a vagina, a penis, and/or an anus of the first person is prevented from transferring to a second person when the second person is orally stimulating the first person by oral contact with the non-permeable material portion.

48. A method, comprising:
a first person wearing the protective garment of claim 1 so that the non-permeable material portion is adjacent a vagina, a penis, a scrotum, and/or an anus of the first person; and
a second person orally stimulating the first person by placing lips and/or a tongue of the second person on the protective garment at the non-permeable material portion;
wherein fluid or other matter from the vagina, the penis, and/or the anus of the first person is prevented from transferring to the second person when the second person is performing the step of orally stimulating the first person.

49. (canceled)
